(19) United States
(12) Patent Application Publication
(10) Pub. No.: US 2025/0340825 A1
(43) Pub. Date: Nov. 6, 2025

(54) ENHANCED FUNGAL MATERIAL

(71) Applicant: Plant Material Limited, Manchester (GB)

(72) Inventors: Peter A. D. Wellham, Manchester (GB); Mihael Jelacevic, Manchester (GB)

(21) Appl. No.: 18/871,098

(22) PCT Filed: Jun. 6, 2023
(86) PCT No.: PCT/GB2023/051475

§ 371 Date: Dec. 2, 2024

(30) Foreign Application Priority Data
Jun. 6, 2022 (GB) 2208266.3

(51) Int. Cl. C12N 1/14; C12R 1/645

(52) U.S. Cl. CPC C12N 1/14; C12R 2001/645

(57) ABSTRACT

The invention relates to enhanced fungal mats having enhanced mechanical properties compared to naturally-occurring fungal mats produced by a fungus or fungi of the same species. The invention also relates to materials comprising said enhanced fungal mat, and to uses of said enhanced fungal mats and materials. The invention further relates to methods of enhancing the mechanical properties of a fungal mat.

### FIELD OF THE INVENTION

The invention relates to enhanced fungal mats. Enhanced fungal mats of the invention have enhanced mechanical properties as compared to naturally-occurring fungal mats produced by a fungus or fungi of the same species. The invention also relates to materials comprising said enhanced fungal mat, and to uses of said enhanced fungal mats and materials. The invention further relates to methods of enhancing the mechanical properties of a fungal mat.

### BACKGROUND OF THE INVENTION

Fungal biomass can be used in the production of a wide range of products. For example, fungal cultures can be used to produce renewable and biodegradable materials with the potential to be used in various applications, e.g. clothing, upholstery, and in medical settings. Fungal cultures also have important roles in the food industry, e.g. in the production of meat substitutes.

The ability to produce materials or other products which contain fungal biomass depends in part on the growth patterns of the fungal strain(s) used, as well as the type of growth substrate (herein “media”) used to grow said fungal strain(s). For some applications, it is desirable to grow fungal culture(s) under conditions which allow the formation of a fungal mat (also known as a “biofilm”). This is typically achieved using solid growth media. However, industrial-scale growth of fungal cultures on solid media is challenging e.g. due to the cost and time associated with sterilising the solid media. In addition, fungal cultures grown on solid media may exhibit reduced homogeneity.

Methods for growing fungal mats in liquid media can be more readily scaled for industrial applications. However, fungal mats grown in liquid media typically exhibit inferior mechanical properties compared to those grown on solid media which, in turn, limits the applicability of fungal mats grown in liquid media. For example, fungal mats grown in liquid media are often more fragile than those produced on solid media, and they may be too thin to provide sufficient structural integrity.

There exists an urgent and unmet need for fungal mats that exhibit enhanced mechanical properties and for industrially applicable methods for producing such enhanced fungal mats.

### SUMMARY OF THE INVENTION

The Inventors have overcome the above challenges by developing a method of enhancing the mechanical properties of a fungal mat. Advantageously, fungal mats of the invention (referred to herein as “enhanced fungal mats”) exhibit enhanced mechanical properties as compared to naturally-occurring fungal mats produced by a fungus or fungi of the same species. In more detail, enhanced fungal mats may exhibit increased thickness, increased tensile strength, increased ultimate load and/or reduced surface adhesion as compared to a naturally-occurring fungal mat produced by a fungus or fungi of the same species. Importantly, the methods of the invention are industrially applicable, not least because their production is not reliant upon the use of solid growth media. The enhanced fungal mats of the invention, and materials comprising said enhanced fungal mats, have a wide range of applications, ranging from the textile and clothing industry to the food industry.

The invention provides an enhanced fungal mat, wherein: (a) the enhanced fungal mat has enhanced mechanical properties compared to a naturally-occurring fungal mat; and (b) the enhanced fungal mat comprises fungus or fungi of the same species as the naturally-occurring fungal mat.

In one embodiment, the enhanced fungal mat has increased tensile strength compared to the naturally-occurring fungal mat. In one embodiment, the tensile strength of the enhanced fungal mat is at least 10% greater than the tensile strength of the naturally-occurring fungal mat. In one embodiment, the enhanced fungal mat has a tensile strength of at least 1000 Pa.

In one embodiment, the enhanced fungal mat has increased ultimate load compared to the naturally-occurring fungal mat. In one embodiment, the ultimate load of the enhanced fungal mat is at least 10% greater than the ultimate load of the naturally-occurring fungal mat. In one embodiment, the enhanced fungal mat has an ultimate load of at least 3 Newtons.

In one embodiment, the enhanced fungal mat has increased thickness compared to the naturally-occurring fungal mat. In one embodiment, the thickness of the enhanced fungal mat is at least 3-fold greater than the thickness of the naturally-occurring fungal mat. In one embodiment, the enhanced fungal mat has a thickness of at least 5 mm.

In one embodiment, the enhanced fungal mat has decreased surface adhesion compared to the naturally-occurring fungal mat. In one embodiment, the surface adhesion of the enhanced fungal mat is at least 10% lower than the surface adhesion of the naturally-occurring fungal mat.

In one embodiment, the enhanced fungal mat further comprises a support material, optionally wherein the support material comprises a fabric.

The invention also provides a material comprising an enhanced fungal mat of the invention.

In one embodiment, the material comprises two or more enhanced fungal mats.

The invention also provides a method of enhancing the mechanical properties of a fungal mat, the method comprising: (a) exposing the fungal mat to unspent media; and/or (b) physically manipulating the fungal mat; to provide an enhanced fungal mat having enhanced mechanical properties relative to the fungal mat. In one embodiment, the method comprises (a) exposing the fungal mat to unspent media; and (b) physically manipulating the fungal mat.

In one embodiment, exposing the fungal mat to unspent media comprises moving the fungal mat to a culture vessel containing unspent media.

In one embodiment, exposing the fungal mat to unspent media comprises adding unspent media to the surface of the fungal mat.

In one embodiment, the method further comprises exposing the fungal mat to additional fungal material, optionally wherein additional fungal material comprises fungal spores and/or fungal hyphae.

In one embodiment, physically manipulating the fungal mat comprises folding the fungal mat, optionally wherein folding the fungal map comprises folding the fungal mat approximately in half.

In one embodiment, physically manipulating the fungal mat comprises flipping the fungal mat.

In one embodiment, physically manipulating the fungal mat comprises contacting the fungal mat with a support material, optionally wherein the support material comprises a fabric.

In one embodiment, physically manipulating the fungal mat comprises combining the fungal mat with one or more additional fungal mat(s) to produce a combined fungal mat.

In one embodiment, the fungal mat and the additional fungal mat each comprise opposing upperside and underside surfaces and wherein combining the fungal mat with the additional fungal mat comprises: (a) contacting the underside surface of the fungal mat with the underside surface of the additional fungal mat; (b) contacting the upperside surface of the fungal mat with the upperside surface of the additional fungal mat; (c) contacting the underside surface of the fungal mat with the upperside surface of the additional fungal mat; or (d) contacting the upperside surface of the fungal mat with the underside surface of the additional fungal mat.

In one embodiment, the method comprises placing a support material between the fungal mat and the additional fungal mat.

In one embodiment, the method further comprises applying force to the outer surfaces of the combined fungal mat.

In one embodiment, the fungal mat is grown in a culture vessel and wherein physically manipulating the fungal mat comprises detaching the edges of the fungal mat from the sides of the culture vessel.

In one embodiment, the method further comprises producing the fungal mat by culturing at least one fungal strain in liquid media. In one embodiment, the method comprises culturing the at least one fungal strain in the presence of a support material and wherein the fungal mat adheres to the support material. In one embodiment, the method comprises inoculating the liquid growth media with fungal spores, fungal hyphae and/or fungal biomass. In one embodiment, culturing the at least one fungal strain in liquid growth media comprises culturing the at least one fungal strain under static conditions. In one embodiment, the liquid growth media is selected from potato dextrose broth and Sabouraud dextrose broth.

The invention also provides an enhanced fungal mat obtainable by the method of the invention. The invention also provides a material comprising an enhanced fungal mat obtained by the method of the invention.

The invention also provides use of an enhanced fungal mat of the invention or a material of the invention as a clothing material or an upholstery material.

The invention also provides use of an enhanced fungal mat of the invention or a material of the invention as a foodstuff.

The invention also provides use of an enhanced fungal mat of the invention or a material of the invention as a medical device.

The invention also provides use of an enhanced fungal mat of the invention or a material of the invention as a dermal or trans-dermal delivery system for metabolite(s).

The invention also provides use of an enhanced fungal mat of the invention or a material of the invention as an insulation material.

The invention also provides use of an enhanced fungal mat of the invention or a material of the invention as a packaging material.

The invention also provides use of an enhanced fungal mat of the invention or a material of the invention as a plastic substitute.

The invention also provides use of an enhanced fungal mat of the invention or a material of the invention as a leather substitute.

The invention also provides use of an enhanced fungal mat of the invention or a material of the invention as a rubber substitute.

The invention also provides a clothing material comprising the enhanced fungal mat of the invention, or the material of the invention.

The invention also provides an upholstery material comprising the enhanced fungal mat of the invention, or the material of the invention.

The invention also provides a foodstuff comprising the enhanced fungal mat of the invention, or the material of the invention.

The invention also provides a medical device comprising the enhanced fungal mat of the invention, or the material of the invention.

The invention also provides a dermal or trans-dermal delivery system for metabolite(s) comprising the enhanced fungal mat of the invention, or the material of the invention.

The invention also provides an insulation material comprising the enhanced fungal mat of the invention, or the material of the invention.

The invention also provides a packaging material comprising the enhanced fungal mat of the invention, or the material of the invention.

The invention also provides a plastic substitute comprising the enhanced fungal mat of the invention, or the material of the invention.

The invention also provides a leather substitute comprising the enhanced fungal mat of the invention, or the material of the invention.

The invention also provides a rubber substitute comprising the enhanced fungal mat of the invention, or the material of the invention.

### BRIEF DESCRIPTION OF THE FIGURES

FIG. 1. Fungal mat thickness represented as fold-change relative to control (A). B-L represent enhanced fungal mats according to the invention (treatment details are provided in Table 1). Error bars show standard error of measurement (SEM).

FIG. 2. (i) Key and structure of a fungal mat depicting the upperside (air exposed/upward-facing) and underside (media exposed/downward-facing) surfaces of the fungal mat. (ii) Experimental set up for production of an initial fungal mat (also referred to herein as a “naturally-occurring fungal mat”).

FIG. 3. Exemplary fungal mat manipulations. (i) The initial fungal mat is flipped thereby exposing the upperside surface to the media. (ii) The initial fungal mat is moved to unspent media. (iii) The initial fungal mat is flipped and moved to unspent media. (iv) Unspent media is added to the upperside surface of the initial fungal mat. (v) A support material and unspent media are added to the upperside surface of the initial fungal mat. (vi) Additional fungal material (e.g. fungal spores, fungal hyphae or other fungal biomass) is added to the upperside surface of the initial fungal mat. (vii) A support material and additional fungal material (e.g. fungal spores, fungal hyphae or other fungal biomass) are added to the upperside surface of the initial fungal mat. ‘(xn)’ indicates that steps can be repeated at least once. Following each manipulation, the fungal mat is typically allowed to grow for a predetermined period of time.

FIG. 4. Exemplary fungal mat manipulation wherein the initial fungal mat is folded in half. Following the manipulation, the fungal mat is typically allowed to grow for a predetermined period of time.

FIG. 5. Exemplary fungal mat manipulations including the formation of a combined fungal mat. Two initial fungal mats are grown separately and then placed together to provide a combined fungal mat with: (i) the uppersides of each fungal mat in contact; (ii) the undersides of each fungal mat in contact; or (iii) the upperside of the first fungal mat in contact with the underside of the second fungal mat. Each of these combined fungal mats may be exposed to a further manipulation such as: (a) the addition of a support material between the fungal mats; (b) the application of force to the outer surface(s) of the combined fungal mat; or (c) a combination of both (a) and (b). Following each manipulation, the combined fungal mat is typically allowed to grow for a predetermined period of time, e.g. in a culture vessel containing liquid media.

FIG. 6. Exemplary fungal mat manipulation methods involving the use of a support material during production of the initial fungal mat. (i) A support material is added to the growth media prior to inoculation of the growth media with the fungal strain. (ii) A support material is directly inoculated with the fungal strain prior to adding the support material to the growth media.

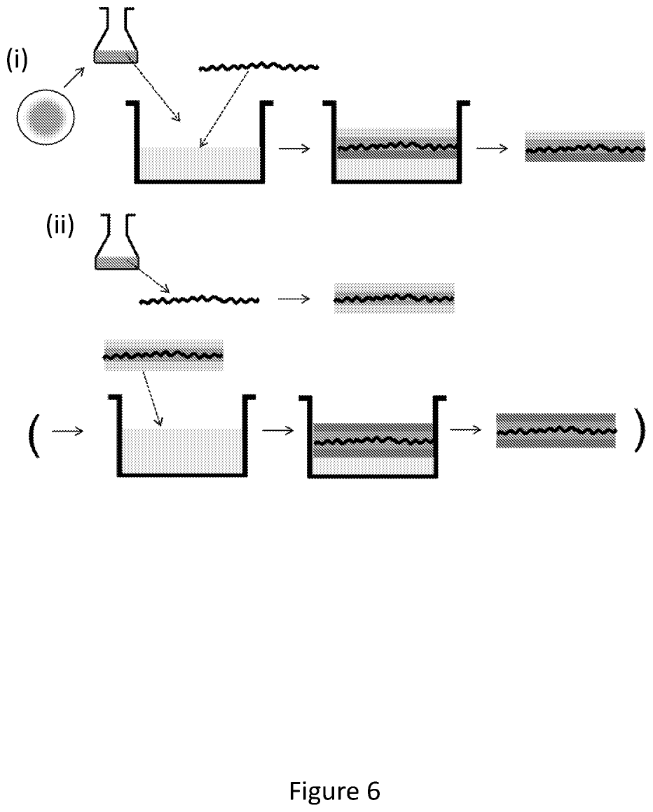

FIG. 7. Exemplary fungal mat manipulation methods involving combining two or more combined fungal mats. As with combined fungal mats, support materials and/or force can be applied after combining the combined fungal mats. The combined fungal mat produced by these manipulation methods may be allowed to grow for a predetermined period of time, e.g. in a culture vessel containing liquid media. (xn) indicates that the combining may be repeated ‘n’ times (n is typically selected from 1, 2, 3, 4, 5, 6, 7, 8, 9 or 10, although a greater number of combinations may also be performed).

### DETAILED DESCRIPTION OF THE INVENTION

The invention provides an enhanced fungal mat wherein the enhanced fungal mat has enhanced mechanical properties compared to a naturally-occurring fungal mat. Enhanced fungal mats of the invention have enhanced mechanical properties as compared to a naturally-occurring fungal mat formed by a fungus or fungi of the same species. The invention also provides methods for producing enhanced fungal mats. The invention further provides a material comprising an enhanced fungal mat of the invention.

Fungal mats typically comprise a large number of fungal cells encased within an extracellular matrix. This extracellular matrix typically includes a combination of macromolecules, such as polysaccharides, proteins, nucleic acids, and lipids, and contributes to various functions including cell and surface adhesion.

The fungal mats described herein are grown on liquid media. It will be understood that references herein to liquid media embrace any fluid media, including liquid and slurry-based media. In some embodiments, the fungal mats described herein are grown by submerged fermentation.

As described herein, a fungal mat that is formed on the surface of liquid growth media typically comprises an “upperside” and an “underside”. Herein, the “upperside” refers to the surface of a fungal mat that is exposed to the air during growth and/or the surface of a fungal mat which faces upwards. The “underside” refers to the surface of the fungal mat that is exposed to the liquid media during growth and/or the surface of a fungal mat which faces downwards. It will be understood that “upward- facing” and “downward-facing”, and the like, refer respectively to the top and bottom surfaces of a fungal mat during static growth on liquid media. The upperside and underside of a fungal mat typically have different properties, e.g. different extracellular matrix compositions. The methods described herein may alter the properties of the upper and undersides of the fungal mat, thereby producing an enhanced fungal mat.

As used herein, an “enhanced fungal mat” is a fungal mat having enhanced mechanical properties as compared to a naturally-occurring fungal mat produced by a fungus or fungi of the same species. Said enhanced mechanical properties include, but are not limited to, increased tensile strength, increased ultimate load, increased thickness, and reduced surface adhesion. Owing to said enhanced mechanical properties, enhanced fungal mats of the invention are advantageously suitable for a wider range of applications than naturally-occurring fungal mats.

As used herein, “naturally-occurring fungal mat” refers to a fungal mat produced by a fungus or fungi that has not been manipulated by a method of the invention. An exemplary naturally-occurring fungal mat is a fungal mat that has been grown under static conditions in liquid media without any physical manipulation. For comparison purposes, the naturally-occurring fungal mat is typically grown for the same period of time and on the same type of growth media as the enhanced fungal mat. In some embodiments, the naturally-occurring fungal mat is a fungal mat that has formed on the surface of liquid media under static growth conditions.

In some embodiments, the enhanced fungal mat has increased ultimate load compared to the naturally-occurring fungal mat. The ultimate load of a material (e.g. a fungal mat) is the maximum force that the material can withstand while being stretched or pulled without breaking. The ultimate load is typically measured in Newtons. The ultimate load of a fungal mat may be measured using any suitable method known in the art, e.g. a universal testing machine (UTM). For example, the ultimate load of a fungal mat may be tested by placing the fungal mat between grips of a UTM and applying a tensile force to the fungal mat until it ruptures. The ultimate load is the ultimate tensile force the fungal mat is able to withstand before rupturing. As used herein, a fungal mat having increased ultimate load relative to a naturally occurring fungal mat is a fungal mat that has a higher ultimate load than the naturally occurring fungal mat.

In some embodiments, the ultimate load of the enhanced fungal mat is at least 10% greater than the ultimate load of the naturally-occurring fungal mat. In some embodiments, the ultimate load of the enhanced fungal mat is at least 15%, at least 20%, at least 25%, at least 30%, at least 35%, at least 40%, at least 45%, at least 50%, at least 60%, at least 70%, at least 80%, at least 90%, or at least 100% greater than the ultimate load of the naturally-occurring fungal mat.

In some embodiments, the ultimate load of the enhanced fungal mat is at least 2-fold greater than the ultimate load of the naturally-occurring fungal mat. In some embodiments, the ultimate load of the enhanced fungal mat is at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold, at least 15-fold, or at least 20-fold greater than the ultimate load of the naturally-occurring fungal mat.

In some embodiments, the enhanced fungal mat has an ultimate load of at least 1, at least 1.5, at least 2, at least 3, at least 4, at least 5, at least 6, at least 7, at least 8, at least 9, at least 10, at least 11, at least 12, at least 13, at least 14, at least 15, at least 20, at least 30, at least 40, at least 50, at least 75, at least 100, at least 150, or at least 200 Newtons. In some embodiments, such as wherein the enhanced fungal mat does not comprise support material, the enhanced fungal mat has an ultimate load of 10-70, 20-70, 30-70, 40-70, 40-60 or 45-55 Newtons.

In some embodiments, the enhanced fungal mat has increased tensile strength compared to the naturally-occurring fungal mat. Tensile strength may be measured by indexing a material's ultimate load to its cross-sectional area. Tensile strength is typically measured in Pascals (Pa). As used herein, a fungal mat having increased tensile strength relative to a naturally occurring fungal mat is a fungal mat that has a higher tensile strength than the naturally occurring fungal mat.

In some embodiments, the tensile strength of the enhanced fungal mat is at least 10% greater than the tensile strength of the naturally-occurring fungal mat. In some embodiments, the tensile strength of the enhanced fungal mat is at least 15%, at least 20%, at least 25%, at least 30%, at least 35%, at least 40%, at least 45%, at least 50%, at least 60%, at least 70%, at least 80%, at least 90%, or at least 100% greater than the tensile strength of the naturally-occurring fungal mat.

In some embodiments, the tensile strength of the enhanced fungal mat is at least 2-fold greater than the tensile strength of the naturally-occurring fungal mat. In some embodiments, the tensile strength of the enhanced fungal mat is at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold, at least 15-fold, or at least 20-fold greater than the tensile strength of the naturally-occurring fungal mat.

In some embodiments, the enhanced fungal mat has a tensile strength of at least 1000, at least 1500, at least 2000, at least 3000, at least 4000, at least 5000, at least 6000, at least 7000, at least 8000, at least 9000, at least 10000, at least 11000, at least 12000, at least 13000, at least 14000, at least 15000, at least 20000, at least 30000, at least 40000, at least 50000, at least 75000, at least 100000, at least 150000, or at least 200000 Pa.

In some embodiments, the enhanced fungal mat has increased thickness compared to the naturally-occurring fungal mat. The thickness of a fungal mat can be measured using any suitable method known in the art, e.g. using a ruler or a thickness gauge. As used herein, the thickness of a fungal mat refers to the distance between the upperside and the underside surfaces of the fungal mat.

In some embodiments, the thickness of the enhanced fungal mat is at least 10% greater than the thickness of the naturally-occurring fungal mat. In some embodiments, the thickness of the enhanced fungal mat is at least 15%, at least 20%, at least 25%, at least 30%, at least 35%, at least 40%, at least 45%, at least 50%, at least 60%, at least 70%, at least 80%, at least 90%, or at least 100% greater than the thickness of the naturally-occurring fungal mat.

In some embodiments, the thickness of the enhanced fungal mat is at least 2-fold greater than the thickness of the naturally-occurring fungal mat. In some embodiments, the thickness of the enhanced fungal mat is at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold, at least 15-fold, or at least 20-fold greater than the thickness of the naturally-occurring fungal mat.

In some embodiments, the enhanced fungal mat has a thickness of at least 3 mm. In some embodiments, the enhanced fungal mat has a thickness of at least 4 mm, at least 5 mm, at least 6 mm, at least 7 mm, at least 8 mm, at least 9 mm, at least 10 mm, at least 11 mm, at least 12 mm, at least 13 mm, at least 14 mm, at least 15 mm, at least 16 mm, at least 17 mm, at least 18 mm, at least 19 mm, at least 20 mm, at least 25 mm, at least 30 mm, at least 35 mm, at least 40 mm, at least 45 mm, at least 50 mm, at least 75 mm or at least 100 mm.

In some embodiments, the enhanced fungal mat has decreased surface adhesion compared to the naturally-occurring fungal mat. Surface adhesion of a fungal mat may be measured using any suitable method known in the art. For example, the surface adhesion of the fungal mat may be measured using a tape test whereby sticky tape, e.g. scotch tape, is put on the surface of the fungal mat and then pulled off. A rating of 0 to 5 (low surface adhesion) is used to characterise the level of surface adhesion wherein a surface adhesion rating of “0” is assigned to the fungal mat when the surface of the fungal mat remains attached to the tape when the tape is removed, and the surface of the fungal mat continues to tear away after removal of the tape; a surface adhesion rating of “1” is assigned when the surface of the fungal mat remains attached to the tape when it is removed but the surface of the fungal mat does not continue to tear after removal of the tape; surface adhesion ratings of “2”, “3” and “4” are intermediate ratings assigned depending on how much of the fungal mat surface remains attached to the tape when it is removed from the surface of the fungal mat; and a surface adhesion rating of “5” is assigned when none of the surface of the fungal mat is removed with the tape and the surface of the fungal mat remains completely undamaged. An enhanced fungal mat may have a greater surface adhesion rating than the naturally-occurring fungal mat. In some embodiments, an enhanced fungal mat has a surface adhesion rating of 4 or 5. In some embodiments, an enhanced fungal mat has a surface adhesion rating of 5.

In some embodiments, the surface adhesion of the enhanced fungal mat is at least 10% lower than the surface adhesion of the naturally-occurring fungal mat. In some embodiments, the surface adhesion of the enhanced fungal mat is at least 15%, at least 20%, at least 25%, at least 30%, at least 35%, at least 40%, at least 45%, at least 50%, at least 60%, at least 70%, at least 80%, at least 90%, or 100% lower than the surface adhesion of the naturally-occurring fungal mat.

In some embodiments, the surface adhesion of the enhanced fungal mat is at least 2-fold lower than the surface adhesion of the naturally-occurring fungal mat. In some embodiments, the surface adhesion of the enhanced fungal mat is at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold, at least 15-fold, or at least 20-fold lower than the surface adhesion of the naturally-occurring fungal mat.

The invention provides a material comprising an enhanced fungal mat. In some embodiments, the material comprises a single enhanced fungal mat. In some embodiments, the material comprises more than one enhanced fungal mat, such as at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, or at least 100 enhanced fungal mats. The enhanced fungal mats may be the same or different. In some embodiments, the material comprises at least 2 enhanced fungal mats produced by different strains of fungus or fungi. In some embodiments, the material comprises at least 2 enhanced fungal mats produced by the same strain of fungus or fungi.

The material or the enhanced fungal mat of the invention may be used in a wide range of applications. In some embodiments, the material or enhanced fungal mat of the invention is used to produce a biofabric e.g. for use in clothing and/or upholstery. The invention therefore provides a biofabric comprising a material and/or enhanced fungal mat of the invention. The invention also provides use of an enhanced fungal mat of the invention or a material of the invention as a clothing material or an upholstery material. A clothing material is a flexible material that is suitable for use in the production of clothes. An upholstery material is a flexible material that is suitable for covering furniture such as sofas and chairs.

In some embodiments, the material or enhanced fungal mat of the invention is used to produce a biomaterial for use in medicine. The invention therefore provides a biomaterial comprising a material and/or enhanced fungal mat of the invention. In some embodiments, the invention provides a medical device comprising a material or enhanced fungal mat of the invention.

In some embodiments, the material or enhanced fungal mat comprises at least one fungal strain which has been modified to express a transgene. In some embodiments, the transgene encodes a medically relevant protein. In some embodiments, the protein is a therapeutic protein, e.g. an antimicrobial protein. Thus, in some embodiments, the material or enhanced fungal mat of the invention is for use as a protein delivery system, e.g. a dermal or trans-dermal delivery system or protein(s). In some embodiments, the material or enhanced fungal mat comprises at least one fungal strain which has been modified to increase the production of metabolite(s) relative to the corresponding wild type fungal strain. In some embodiments, the metabolite is a medically relevant metabolite. In some embodiments, the metabolite is a therapeutic metabolite, e.g. an antimicrobial metabolite. Thus, in some embodiments, the material or enhanced fungal mat of the invention is for use as a metabolite delivery system, e.g. a dermal or trans-dermal delivery system for metabolite(s). In some embodiments, the protein is an industrially relevant protein. In some embodiments, the metabolite is an industrially relevant metabolite. The fungal strain may be modified using any suitable method, e.g. by genetic engineering, site directed mutagenesis or directed evolution.

In some embodiments, the material or enhanced fungal mat of the invention is used to produce an insulation material, e.g. for use in buildings. The invention therefore provides an insulation material comprising a material and/or enhanced fungal mat of the invention.

In some embodiments, the material or enhanced fungal mat of the invention is used in the production of a foodstuff. The foodstuff may be a meat substitute. The invention therefore provides a foodstuff comprising a material and/or enhanced fungal mat of the invention.

The invention provides a method of enhancing the mechanical properties of a fungal mat, the method comprising: (a) exposing the fungal mat to unspent media; and/or (b) physically manipulating the fungal mat; to provide an enhanced fungal mat having enhanced mechanical properties. Following exposure to unspent media and/or physical manipulation, the fungal mat is typically allowed to grow for a predetermined period of time, such as until the fungal mat achieves a desired thickness. The Inventors have discovered that fungal mat structure may be modified by the methods of the invention, and that such structural modifications achieve improvements in various mechanical properties, e.g. thickness, tensile strength, ultimate load, and/or surface adhesion, of the fungal mat. Without wishing to be bound by theory, the Inventors believe that these improved mechanical properties are achieved via changes in gene expression, e.g. at the transcriptional and/or translational level, of the fungus/fungi that are induced by the methods described herein.

The invention also provides an enhanced fungal mat obtained by a method of the invention.

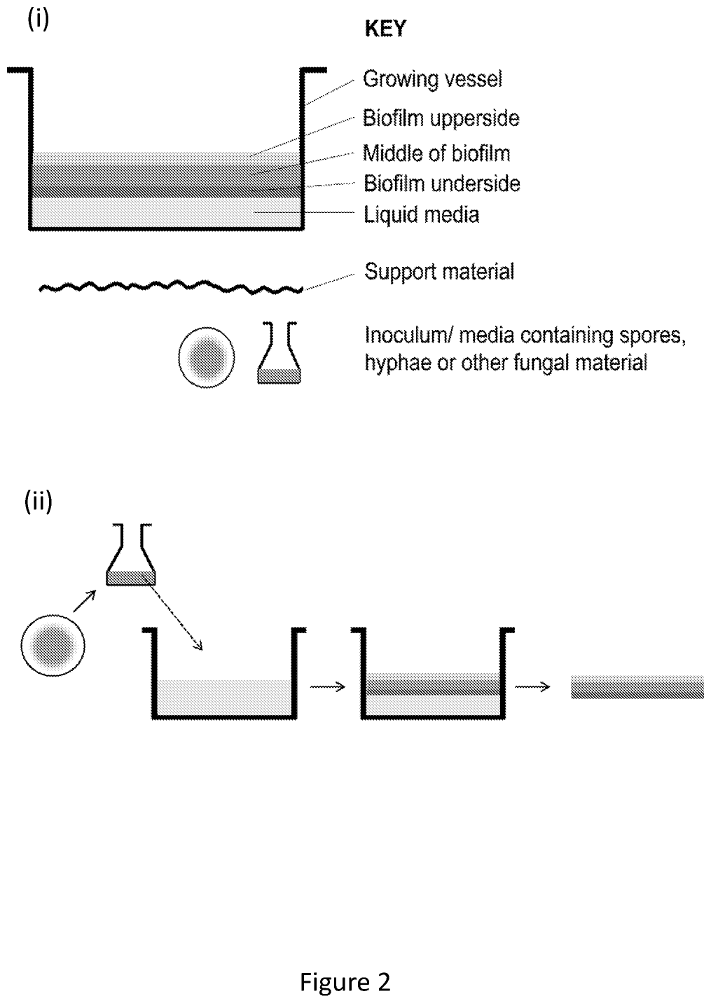

The “initial fungal mat” is typically a fungal mat that has been grown in liquid media under static conditions (e.g. as depicted in FIG. 2(ii)). An “initial fungal mat” which does not undergo further manipulations typically corresponds to the naturally-occurring fungal mat.

Herein, growth “in” liquid media typically refers to growth on the surface of liquid media, or substantially at the surface of liquid media.

In some embodiments, fungal mats are grown for a “predetermined period of time”. In some embodiments, the predetermined period of time is the period of time required for fungal strain(s) to establish a visible fungal mat. In some embodiments, the predetermined period of time is the period of time required for the fungal mat to reach a desired thickness. The skilled person will appreciate that the predetermined period of time may routinely be optimised according to the growth rate(s) of particular fungal strain(s).

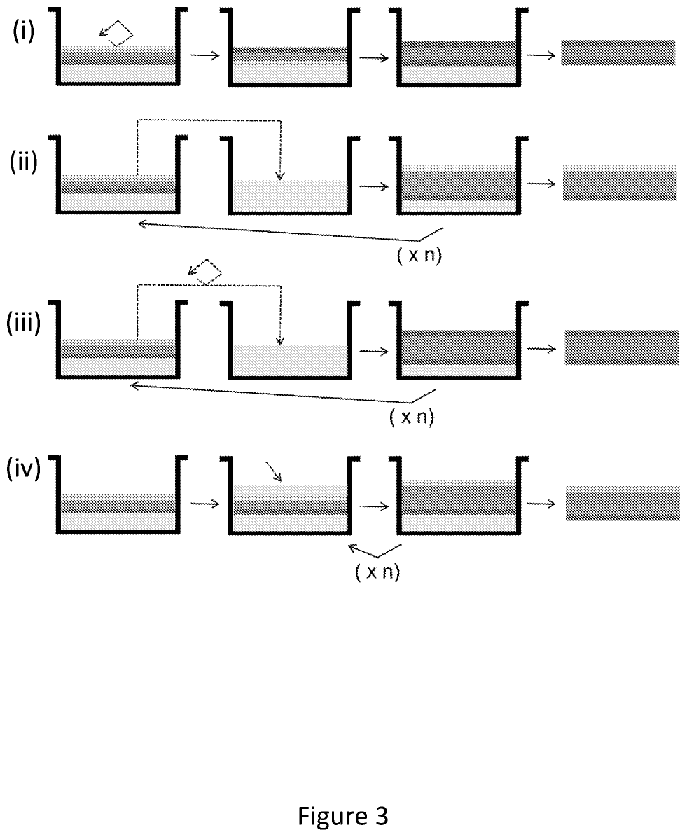

As used herein, “unspent media” typically refers to growth media that has not previously been used to support microbial growth, e.g. fungal growth. In some embodiments, exposing a fungal mat to unspent media comprises moving the fungal mat to a culture vessel containing unspent media (e.g. as depicted in FIG. 3(ii)). In some embodiments, exposing a fungal mat to unspent media comprises replacing the growth media in which a fungal mat has grown with unspent media. In some embodiments, exposing a fungal mat to unspent media comprises adding unspent growth media to the culture vessel in which the fungal mat has been grown. In some embodiments, unspent media has previously been used to support microbial growth, but retains the capacity to support further microbial growth. In some embodiments, wherein unspent media has previously been used to support microbial growth, the unspent media has been sterilised (e.g. autoclaved) prior to use in a method of the invention. In some embodiments, wherein unspent media has previously been used to support microbial growth, the unspent media was not sterilised subsequent to supporting microbial growth and prior to use in a method of the invention.

In some embodiments, exposing the fungal mat to unspent media comprises adding unspent media to the upperside surface of the fungal mat (e.g. as depicted in FIG. 3(iv)). In some embodiments, the fungal mat may be submerged so that both the upperside and underside surfaces of the fungal mat are exposed to media. In some embodiments, a sufficient volume of unspent media is added to the surface of the fungal mat to detach the edges of the fungal mat from the sides of the culture vessel. In some embodiments, exposing the fungal mat to unspent media comprises: (i) replacing the growth media in the culture vessel where the fungal mat has been grown with unspent media; and (ii) adding unspent media to the upperside surface of the fungal mat. In some embodiments, exposing the fungal mat to unspent media comprises: (i) moving the fungal mat to a culture vessel containing unspent media; and (ii) adding unspent media to the upperside surface of the fungal mat.

In some embodiments, the method further comprises exposing the fungal mat to additional fungal material (e.g. as depicted in FIG. 3(vi)). In some embodiments, the additional fungal material is selected from fungal spores, fungal hyphae, or other fungal biomass. The fungal biomass may be a fungal colony isolated from solid growth media, e.g. an agar plate. In some embodiments, the fungal mat is exposed to unspent media comprising additional fungal material. As used herein, additional fungal material includes any reproductive form of fungal biomass, including, but not limited to, spores and hyphae.

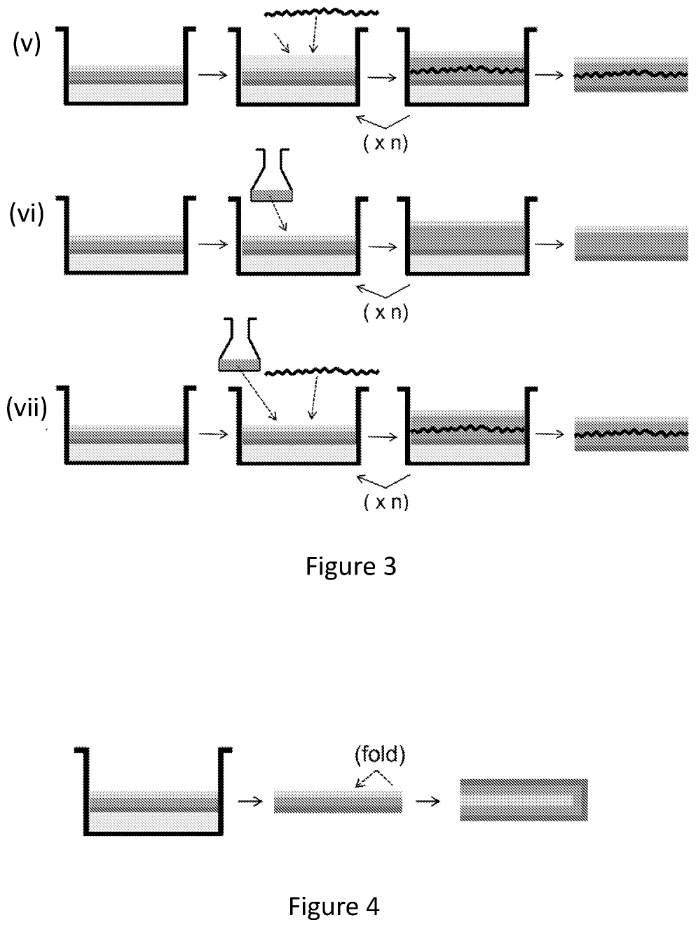

As used herein, physically manipulating the fungal mat means that the fungal mat is moved in some way, e.g. it is moved, displaced and/or rotated. In some embodiments, physically manipulating the fungal mat comprises folding the fungal mat. When the fungal mat is folded, parts of the same surface of the fungal mat come into contact with each other. In some embodiments, folding the fungal mat comprises folding the fungal mat approximately in half, e.g. along an approximate line of symmetry (e.g. as depicted in FIG. 4(i)). In some embodiments, folding the fungal mat comprises folding the edges of the fungal mat towards the centre of the fungal mat.

In some embodiments, physically manipulating the fungal mat comprises flipping the fungal mat. When the fungal mat is flipped, the surfaces are typically reversed so that the upperside surface becomes the underside surface (e.g. as depicted in FIG. 3(i)). In some embodiments, the fungal mat is flipped and then exposed to unspent media (e.g. as depicted in FIG. 3(iii)).

In some embodiments, physically manipulating the fungal mat comprises combining the fungal mat with one or more additional fungal mat(s) to produce a combined fungal mat. The combined fungal is then typically grown for a predetermined period of time, such as until the combined fungal mat achieves a desired thickness. As used herein, a “combined fungal mat” is a composite fungal mat formed by the amalgamation of at least two fungal mats that have been grown separately.

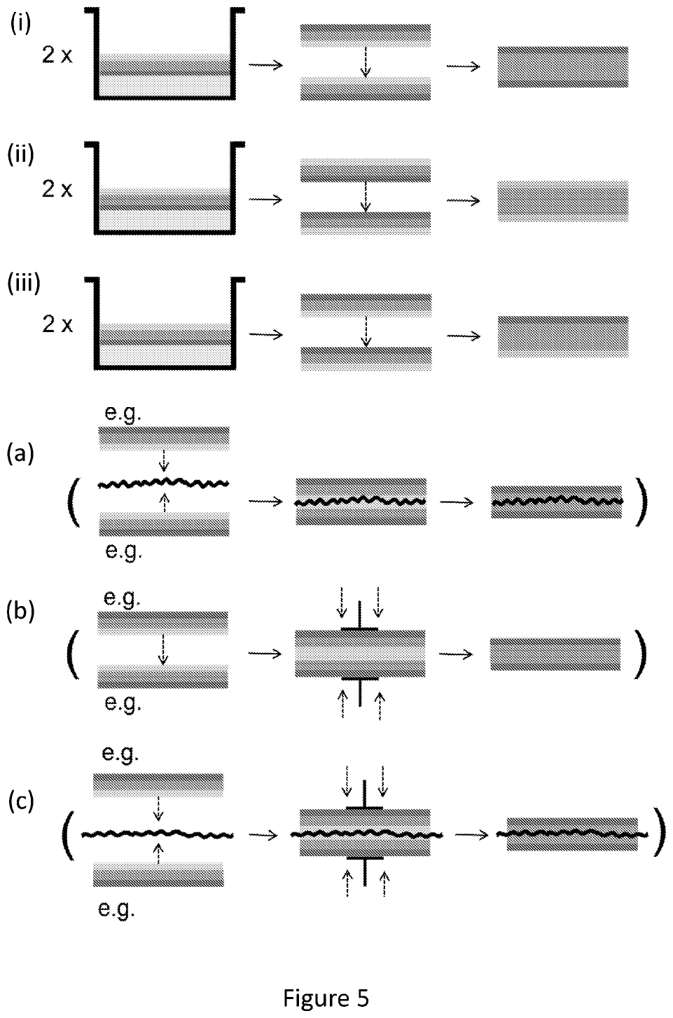

In some embodiments, the fungal mat and the additional fungal mat are combined in opposite orientations e.g. the method comprises contacting: (a) contacting the underside surface of the fungal mat with the underside surface of the additional fungal mat (e.g. as depicted in FIG. 5(ii)); or (b) contacting the upperside surface of the fungal mat with the upperside surface of the additional fungal mat (e.g. as depicted in FIG. 5(i)).

In some embodiments, the fungal mat and the additional fungal mat are combined in the same orientation so that opposing surfaces are in contact e.g. (a) contacting the underside surface of the fungal mat with the upperside surface of the additional fungal mat; and/or (b) contacting the upperside surface of the fungal mat with the underside surface of the additional fungal mat (e.g. as depicted in FIG. 5(iii)).

In some embodiments, the method comprises combining the fungal mat with at least 2, at least 3, at least 4, at least 5, at least 6, at least 7, at least 8, at least 9, at least 10, at least 15, or at least 20 additional fungal mats. In some embodiments, the one or more additional fungal mat(s) is an initial fungal mat as described herein. In some embodiments, the one or more additional fungal mat(s) is an enhanced fungal mat as described herein. In some embodiments, additional fungal mats comprise one or more initial fungal mat(s) and one or more enhanced fungal mat(s).

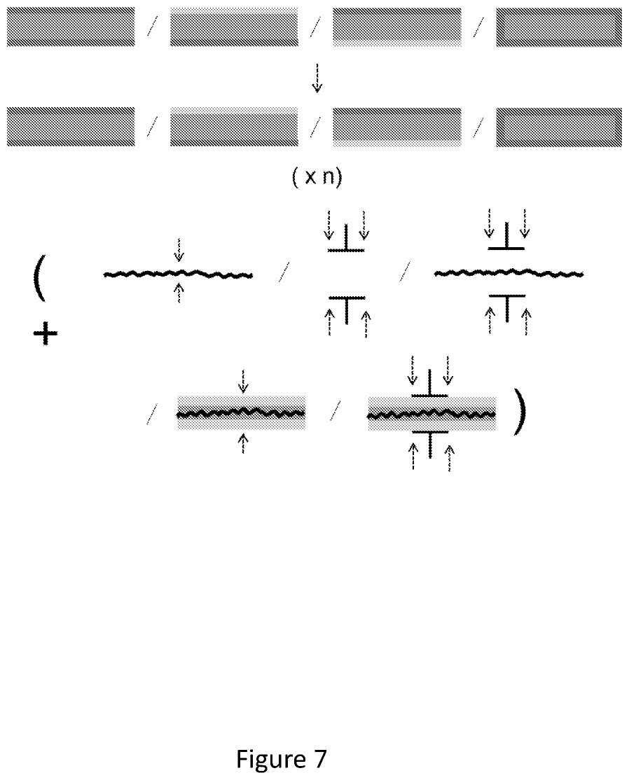

In some embodiments, the method further comprises combining a combined fungal mat with at least a second combined fungal mat (e.g. as depicted in FIG. 7).

In some embodiments, physically manipulating the fungal mat comprises folding the fungal mat and then combining the folded fungal mat with an additional fungal mat to produce a combined fungal mat. In some embodiments, the additional fungal mat is positioned to cover the edges of the folded fungal mat. In some embodiments, the additional fungal mat is positioned to cover the edges of the folded fungal mat and excess portions of the additional fungal mat are removed (e.g. as shown in FIG. 4(ii)).

In some embodiments, the method further comprises applying force to the combined fungal mat (e.g. as depicted in FIGS. 5(b) and (c)). Force is typically used to press the fungal mats together and is generally applied before growing the combined fungal mat, typically for a predetermined period of time, such as until the combined fungal mat achieves a desired thickness. In some embodiments, force is applied by placing the combined fungal mat in a clamp. In some embodiments, force is applied by placing a weight on a surface of the combined fungal mat.

The force may be measured relative to the surface area of the fungal mat, i.e. the pressure applied to the fungal mat. The pressure applied to the fungal mat is typically at least 100, at least 200, at least 300, at least 400, at least 500, at least 600, at least 700, at least 800, at least 900, or at least 1000 pascals.

In some embodiments, physically manipulating the fungal mat comprises contacting the fungal mat with a support material. In some embodiments, the support material comprises a fabric. In some embodiments, the method comprises placing the support material in contact with the upperside surface of the fungal mat. In some embodiments, the method comprises: (i) placing the support material in contact with the upperside surface of the fungal mat; and (ii) adding unspent media to the upperside surface of the fungal mat (e.g. as depicted in FIG. 3(v)).

In some embodiments, the method comprises: (i) placing the support material in contact with the upperside surface of the fungal mat; and (ii) exposing the fungal mat to additional fungal material (e.g. as depicted in FIG. 3(vii)).

In some embodiments, the method comprises placing the support material between the fungal mat and the additional fungal mat when forming a combined fungal mat as described above (e.g. as depicted in FIG. 5(a)).

In some embodiments, physically manipulating the fungal mat comprises detaching the fungal mat from the culture vessel in which it has been grown, e.g. detaching the edges of the fungal mat from the sides of the culture vessel.

In some embodiments, the method comprises: (i) flipping the fungal mat; and (ii) placing a support material on top of the fungal mat. In some embodiments, the method comprises: (i) flipping the fungal mat; and (ii) placing a second fungal mat on top of the first fungal mat to provide a combined fungal mat. In some embodiments, the method comprises: (i) flipping the fungal mat; and (ii) placing a second flipped fungal mat on top of the first fungal mat to provide a combined fungal mat. In some embodiments, the method comprises: (i) flipping the fungal mat; (ii) placing a support material on top of the fungal mat; and (iii) placing a second fungal mat on top of the support material to provide a combined fungal mat. In some embodiments, the method comprises: (i) flipping the fungal mat; (ii) placing a support material on top of the fungal mat; and (iii) placing a second flipped fungal mat on top of the support material to provide a combined fungal mat. In some embodiments, the method comprises flipping the fungal mat. In some embodiments, the method comprises transferring the fungal mat to a vessel containing unspent media. In some embodiments, the method comprises: (i) slipping the fungal mat; and (ii) transferring the fungal mat to a vessel containing unspent media. In each of these embodiments, the method further comprises growing the fungal mat or combined fungal mat, typically for a predetermined period of time, such as until the fungal mat or combined fungal mat reaches a desired thickness.

In some embodiments, the method further comprises producing the fungal mat by culturing fungal strain(s) in liquid media. The method may comprise inoculating liquid media with additional fungal material, such as fungal hyphae and/or spores, and incubating the culture until a visible fungal mat has formed. In some embodiments, the culture is incubated until a fungal mat having a desired thickness has formed. A desired thickness may be at least 1 mm, at least 2 mm, at least 3 mm, at least 4 mm, or at least 5 mm. In some embodiments, the culture is incubated for a predetermined period of time, e.g. about 4 days, about 7 days, about 2 weeks, about 3 weeks, about 4 weeks, about 5 weeks, about 6 weeks, about 8 weeks, about 10 weeks, or about 12 weeks.

In some embodiments, the method comprises culturing the fungal strain(s) in the presence of a support material. In some embodiments, as the fungal mat grows, it attaches to said support material. In some embodiments, the method comprises inoculating a support material directly with fungal strain(s) and then adding the support material to growth media.

In some embodiments, the method further comprises drying the enhanced fungal mat. In some embodiments, the enhanced fungal mat is dried by heating the enhanced fungal mat to at least 30° C., at least 35° C., at least 40° C., at least 45° C., at least 50° C., at least 55° C., at least 60° C., at least 65° C., at least 70° C., at least 75° C., at least 80° C., at least 85° C., at least 90° C., at least 95° C., or at least 100° C. In some embodiments, the enhanced fungal mat is dried by heating the enhanced fungal mat for at least 30 minutes, at least 1 hour, at least 2 hours, at least 3 hours, at least 4 hours, at least 6 hours, at least 8 hours, at least 10 hours, at least 12 hours, at least 24 hours, or at least 48 hours.

The fungal mat manipulation steps described above may be combined in any way. Each fungal mat manipulation step described herein may be repeated at least once, at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, or at least 20 times.

A support material as described herein is any suitable material to which fungal mats can attach during growth. Exemplary support materials include, but are not limited to, fabrics made from cotton, hemp, polyester, nylon, denim, wool, and/or silk. Exemplary support materials also include foodstuffs, such as meat substitutes and/or tofu.

The fungal strain may be any fungal strain, or combination of fungal strains, that is capable of forming a fungal mat. In some embodiments, the fungus is an Ascomycota or a Basidiomycota. In some embodiments, the fungus is a member of the Clavicipitaceae family or the Trichocomaceae family. In some embodiments, the fungus is a member of a genus selected from Cordyceps, Metacordyceps, Pleurotus, Hericium, Grifola, Fistulina, Fomes, Fomitopsis, Inonotus, Trametes Ganoderma, Aspergillus, Penicillium, Chrysosporium, Myceliophthora, Trichoderma, Acremonium, and Fusarium. In some embodiments, the fungus is selected from Cordyceps sensu lato, Pleurotus ostreatus, Pleurotus eryngii, Hericium erinaceus, Grifola frondosa, Fistulina hepatica, Fomes fomentarius, Fomitopsis pinicola, Inonotus obliquus, Trametes versicolor, Ganoderma applanatum, and Ganoderma lucidum.

In some embodiments, the enhanced fungal mat is an enhanced Cordyceps mat. In some embodiments, the enhanced fungal mat is an enhanced Aspergillus mat. In some embodiments, the enhanced fungal mat is an enhanced Pleurotus ostreatus mat. In some embodiments, the enhanced fungal mat is an enhanced Pleurotus eryngii mat. In some embodiments, the enhanced fungal mat is an enhanced Hericium erinaceus mat. In some embodiments, the enhanced fungal mat is an enhanced Grifola frondose mat. In some embodiments, the enhanced fungal mat is an enhanced Fistulina hepatica mat. In some embodiments, the enhanced fungal mat is an enhanced Fomes fomentarius mat. In some embodiments, the enhanced fungal mat is an enhanced Fomitopsis pinicola mat. In some embodiments, the enhanced fungal mat is an enhanced Inonotus obliquus mat. In some embodiments, the enhanced fungal mat is an enhanced Trametes versicolor mat. In some embodiments, the enhanced fungal mat is an enhanced Ganoderma applanatum mat. In some embodiments, the enhanced fungal mat is an enhanced Ganoderma lucidum mat.

The growth media may be any suitable growth media and is typically liquid growth media. In some embodiments, the growth media is selected from potato dextrose broth, Sabouraud dextrose broth, Czapek-Dox broth, and malt extract broth.

The fungal mat is typically grown for a predetermined period of time. In some embodiments, the predetermined period of time is e.g. about 4 days, about 7 days, about 2 weeks, about 3 weeks, about 4 weeks, about 5 weeks, about 6 weeks, about 8 weeks, about 10 weeks, or about 12 weeks. In some embodiments, the predetermined period of time is the period of time required for the fungal mat to grow to a desired thickness e.g. at least 1 mm, at least 2 mm, at least 3 mm, at least 4 mm, at least 5 mm, at least 6 mm, at least 7 mm, at least 8 mm, at least 9 mm, or at least 10 mm.

The fungal mat is typically grown under static conditions.

The fungal mat is typically grown at between 15° C. and 60° C., at between 15° C. and 50° C., at between 15° C. and 45° C., at between 15° C. and 40° C., at between 15° C. and 35° C., at between 15° C. and 30° C., at between 20° C. and 60° C., at between 20° C. and 50° C., at between 20° C. and 45° C., at between 20° C. and 40° C., at between 20° C. and 35° C., at between 20° C. and 30° C., at between 30° C. and 60° C., at between 30° C. and 50° C., at between 30° C. and 45° C., at between 30° C. and 40° C., or at between 30° C. and 35° C.

### EXAMPLES

The invention will be further clarified by the following examples, which are intended to be purely exemplary of the invention and are in no way limiting.

### Example 1

### Impact of Manipulation Methods on Fungal Mat Thickness

A fungal mat was produced by inoculating 80 ml of potato dextrose broth with a Cordyceps sp. spore suspension comprising approximately 2 million spores in an Erlenmeyer flask. The culture was incubated under dark, static conditions at 24° C. for 2 weeks to form a naturally-occurring fungal mat, also referred to herein as an initial fungal mat. For the control, designated “A” below, the initial fungal mat was grown for an additional 2 weeks without any additional manipulation.

The initial fungal mat was then exposed to various manipulations methods of the invention as described in Table 1. Experiments were conducted in triplicate.

Following the manipulation methods described in Table 1, the initial fungal mat and the enhanced fungal mats (designated ‘B’-‘L’) were dried at 45° C. for several hours, and thickness was measured.

TABLE 1
Fungal mat manipulations methods

| Name | Manipulation method |
|---|---|
| A | Liquid broth is inoculated with fungal biomass and incubated |
| | for 4 weeks to provide a fungal mat. No further manipulation |
| | is performed. |
| B | Liquid broth is inoculated with fungal biomass and incubated |
| | for 2 weeks to provide an initial fungal mat; the fungal mat |
| | is then flipped, and a sterile cotton support material is placed |
| | on top; the fungal mat is then grown for a further 2 weeks |
| C | Two initial fungal mats are grown as in B; the first fungal |
| | mat is then flipped, and the second fungal mat is placed on |
| | top of the first fungal mat to provide a combined fungal mat; |
| | the combined fungal mat is then grown for a further 2 weeks - |
| | the second (top) fungal mat is isolated for further analysis |
| D | As above for C - the first (bottom) fungal mat is isolated |
| | for further analysis |
| E | Two initial fungal mats are grown as in B; both fungal mats |
| | are then flipped, and the first fungal mat is placed on top |
| | of the second fungal mat to provide a combined fungal mat; |
| | the combined fungal mat is then grown for a further 2 weeks - |
| | the top fungal mat is isolated for further analysis |
| F | As above for E - the bottom fungal mat is isolated for further |
| | analysis |
| G | Two initial fungal mats are grown as in B; the first fungal |
| | mat is flipped, and a sterile cotton support material is placed |
| | on top of the first fungal mat; the second fungal mat is placed |
| | on top of the support material to provide a combined fungal mat; |
| | the combined fungal mat is then grown for a further 2 weeks - |
| | the top fungal mat is isolated for further analysis |
| H | As above for G - the bottom fungal mat is isolated for further |
| | analysis |
| I | Two initial fungal mats are grown as in B; both fungal mats |
| | are then flipped, a sterile cotton support material is placed |
| | on top of the first fungal mat and the second fungal mat is |
| | placed on top of the support material to provide a combined |
| | fungal mat; the combined fungal mat is then grown for a further |
| | 2 weeks - the top fungal mat is isolated for further analysis |
| J | An initial fungal mat is grown as in B; the fungal mat is then |
| | flipped, and grown for a further 2 weeks |
| K | An initial fungal mat is grown as in B; the fungal mat is then |
| | transferred to a new vessel containing unspent media and grown |
| | for a further 2 weeks |
| L | An initial fungal mat is grown as in B; the fungal mat is then |
| | flipped and transferred to a new vessel containing unspent |
| | media; the fungal mat is then grown for a further 2 weeks |

TABLE 2
Thickness of fungal mats provided as fold-
change relative to the control mat

| | A | B | C | D | E | F | G | H | I | J | K | L |
|---|---|---|---|---|---|---|---|---|---|---|---|---|
| Average | 1 | 5.3 | 16 | 5.3 | 8 | 5.3 | 5 | 5 | 5.3 | 3.7 | 7.7 | 8 |
| Rep 1 | 1 | 6 | 20 | 6 | 6 | 4 | 5 | 5 | 5 | 3 | 7 | 8 |
| Rep 2 | 1 | 5 | 14 | 6 | 10 | 6 | 5 | 5 | 5 | 4 | 8 | 7 |
| Rep 3 | 1 | 5 | 14 | 4 | 8 | 6 | 5 | 5 | 6 | 4 | 8 | 9 |

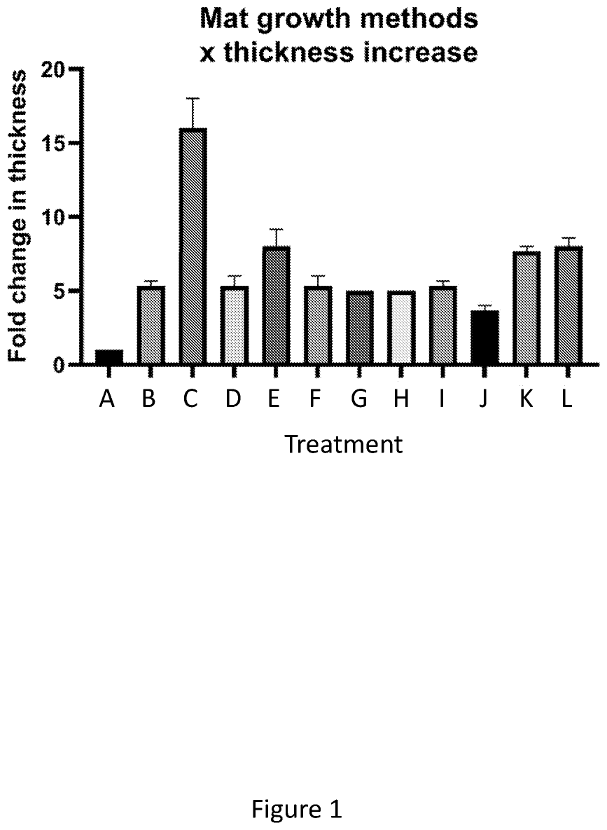

Each of the manipulation methods described in Table 1 produced enhanced fungal mats with significantly improved thickness compared to the initial fungal mat (A). The mean increase in thickness ranged from 3.7-fold to 16-fold relative to the initial fungal mat (see Table 2 and FIG. 1).

These data clearly demonstrate that the manipulation methods of the invention significantly improve the mechanical properties (e.g. thickness) of initial fungal mats thereby providing enhanced fungal mats that can be used in a range of applications.

### Example 2

### Production of Fungal Based Meat Substitutes

An edible fungal strain, or combination of edible fungal strains, is selected from Pleurotus ostreatus, Pleurotus eryngii, Hericium erinaceus, Grifola frondosa, Fistulina hepatica, Fomes fomentarius, Fomitopsis pinicola, Inonotus obliquus, Trametes versicolor, Ganoderma applanatum, and/or Ganoderma lucidum. The fungal strain(s) are grown in liquid media under static conditions until a visible fungal mat has formed.

The fungal mat is then exposed to one of the manipulation methods described in Table 1 to provide an enhanced fungal mat. The wet thickness of the enhanced fungal mat is then measured.

The manipulations methods of the invention significantly improve the thickness of the fungal mats thereby providing edible fungal mats that can be used as meat substitutes.

1. A method of enhancing the mechanical properties of a fungal mat grown in liquid media, the method comprising:
(a) exposing the fungal mat to unspent media; and/or
(b) physically manipulating the fungal mat;
to provide an enhanced fungal mat having enhanced mechanical properties relative to the fungal mat.

2. The method of claim 1, wherein the method comprises:
(a) exposing the fungal mat to unspent media; and
(b) physically manipulating the fungal mat.

3. The method of claim 1 or claim 2, wherein the method comprises exposing the fungal mat to unspent media and physically manipulating the fungal mat by flipping the fungal mat, wherein: (i) the fungal mat is flipped before being exposed to unspent media; and/or (ii) the fungal mat is flipped after being exposed to unspent media.

4. The method of any preceding claim, wherein the method comprises exposing the fungal mat to unspent media and physically manipulating the fungal mat by placing an additional fungal mat on the upperside of the fungal mat.

5. The method of any preceding claim, wherein exposing the fungal mat to unspent media comprises moving the fungal mat to a culture vessel containing unspent media.

6. The method of any preceding claim, wherein exposing the fungal mat to unspent media comprises adding unspent media to the surface of the fungal mat.

7. The method of any preceding claim, wherein the method further comprises exposing the fungal mat to additional fungal material, optionally wherein additional fungal material comprises fungal spores and/or fungal hyphae.

8. The method of any preceding claim, wherein physically manipulating the fungal mat comprises folding the fungal mat, optionally wherein folding the fungal map comprises folding the fungal mat approximately in half.

9. The method of any preceding claim, wherein physically manipulating the fungal mat comprises flipping the fungal mat.

10. The method of any preceding claim, wherein physically manipulating the fungal mat comprises contacting the fungal mat with a support material, optionally wherein the support material comprises a fabric.

11. The method of any preceding claim, wherein physically manipulating the fungal mat comprises combining the fungal mat with one or more additional fungal mat(s) to produce a combined fungal mat.

12. The method of claim 11, wherein the fungal mat and the additional fungal mat each comprise opposing upperside and underside surfaces and wherein combining the fungal mat with the additional fungal mat comprises:
(a) contacting the underside surface of the fungal mat with the underside surface of the additional fungal mat;
(b) contacting the upperside surface of the fungal mat with the upperside surface of the additional fungal mat;
(c) contacting the underside surface of the fungal mat with the upperside surface of the additional fungal mat; or
(d) contacting the upperside surface of the fungal mat with the underside surface of the additional fungal mat.

13. The method of claim 11 or claim 12, wherein the method comprises placing a support material between the fungal mat and the additional fungal mat.

14. The method of any of claims 11-13, wherein the method further comprises applying force to the outer surfaces of the combined fungal mat.

15. The method of any preceding claim, wherein the fungal mat is grown in a culture vessel and wherein physically manipulating the fungal mat comprises detaching the edges of the fungal mat from the sides of the culture vessel.

16. The method of preceding claim, wherein the method further comprises producing the fungal mat by culturing at least one fungal strain in liquid media.

17. The method of claim 16, wherein the method comprises culturing the at least one fungal strain in the presence of a support material and wherein the fungal mat adheres to the support material.

18. The method of claim 16 or claim 17, wherein the method comprises inoculating the liquid growth media with fungal spores, fungal hyphae and/or fungal biomass.

19. The method of any of claims 16-18, wherein culturing the at least one fungal strain in liquid growth media comprises culturing the at least one fungal strain under static conditions.

20. The method of any of claims 16-19, wherein the liquid growth media is selected from potato dextrose broth and Sabouraud dextrose broth.

21. An enhanced fungal mat obtainable by the method of any preceding claim.

22. An enhanced fungal mat, wherein:
(a) the enhanced fungal mat has enhanced mechanical properties compared to a naturally-occurring fungal mat; and
(b) the enhanced fungal mat comprises fungus or fungi of the same species as the naturally-occurring fungal mat.

23. The enhanced fungal mat of claim 22, wherein the enhanced fungal mat has increased tensile strength compared to the naturally-occurring fungal mat.

24. The enhanced fungal mat of claim 23, wherein the tensile strength of the enhanced fungal mat is at least 10% greater than the tensile strength of the naturally-occurring fungal mat.

25. The enhanced fungal mat of any one of claims 22-24, wherein the enhanced fungal mat has a tensile strength of at least 1000 Pa.

26. The enhanced fungal mat of any one of claims 22-25, wherein the enhanced fungal mat has an ultimate load of at least 3 Newtons.

27. The enhanced fungal mat of any one of claims 22-26, wherein the enhanced fungal mat has increased thickness compared to the naturally-occurring fungal mat.

28. The enhanced fungal mat of claim 27, wherein the thickness of the enhanced fungal mat is at least 3-fold greater than the thickness of the naturally-occurring fungal mat.

29. The enhanced fungal mat of any one of claims 22-28, wherein the enhanced fungal mat has a thickness of at least 5 mm.

30. The enhanced fungal mat of any one of claims 22-29, wherein the enhanced fungal mat has decreased surface adhesion compared to the naturally-occurring fungal mat.

31. The enhanced fungal mat of claim 30, wherein the surface adhesion of the enhanced fungal mat is at least 10% lower than the surface adhesion of the naturally-occurring fungal mat.

32. The enhanced fungal mat of any one of claims 22-31, wherein the enhanced fungal mat further comprises a support material, optionally wherein the support material comprises a fabric.

33. A material comprising the enhanced fungal mat of any one of claims 21-32.

34. The material of claim 33, wherein the material comprises two or more enhanced fungal mats.

35. The enhanced fungal mat of any of claims 22-32 or a material of claim 33 or claim 34, wherein the enhanced fungal mat is obtained by the method of any one of claims 1-20.

36. Use of an enhanced fungal mat of any of claim 21-32 or 35, or a material of any of claims 33-35 as a clothing material or an upholstery material.

37. Use of an enhanced fungal mat of any of claim 21-32 or 35, or a material of any of claims 33-35 as a foodstuff.

38. Use of an enhanced fungal mat of any of claim 21-32 or 35, or a material of any of claims 33-35 as a medical device.

39. Use of an enhanced fungal mat of any of claim 21-32 or 35, or a material of any of claims 33-35 as a dermal or trans-dermal delivery system for metabolite(s).

40. Use of an enhanced fungal mat of any of claim 21-32 or 35, or a material of any of claims 33-35 as an insulation material.

41. Use of an enhanced fungal mat of any of claim 21-32 or 35, or a material of any of claims 33-35 as a packaging material.

42. Use of an enhanced fungal mat of any of claim 21-32 or 35, or a material of any of claims 33-35 as a plastic substitute.

43. Use of an enhanced fungal mat of any of claim 21-32 or 35, or a material of any of claims 33-35 as a leather substitute.

44. Use of an enhanced fungal mat of any of claim 21-32 or 35, or a material of any of claims 33-35 as a rubber substitute.

45. A clothing material comprising the enhanced fungal mat of any of claim 21-32 or 35, or the material of any of claims 33-35.

46. An upholstery material comprising the enhanced fungal mat of any of claim 21-32 or 35, or the material of any of claims 33-35.

47. A foodstuff comprising the enhanced fungal mat of any of claim 21-32 or 35, or the material of any of claims 33-35.

48. A medical device comprising the enhanced fungal mat of any of claim 21-32 or 35, or the material of any of claims 33-35.

49. A dermal or trans-dermal delivery system for metabolite(s) comprising the enhanced fungal mat of any of claim 21-32 or 35, or the material of any of claims 33-35.

50. An insulation material comprising the enhanced fungal mat of any of claim 21-32 or 35, or the material of any of claims 33-35.

51. A packaging material comprising the enhanced fungal mat of any of claim 21-32 or 35, or the material of any of claims 33-35.

52. A plastic substitute comprising the enhanced fungal mat of any of claim 21-32 or 35, or the material of any of claims 33-35.

53. A leather substitute comprising the enhanced fungal mat of any of claim 21-32 or 35, or the material of any of claims 33-35.

54. A rubber substitute comprising the enhanced fungal mat of any of claim 21-32 or 35, or the material of any of claims 33-35.
